(19) United States
(12) Patent Application Publication
(10) Pub. No.: US 2025/0376771 A1
(43) Pub. Date: Dec. 11, 2025

(54) CLEAN HYDROGEN (H2) PRODUCTION FROM A WATER DESALINATION PLANT

(71) Applicant: Abdulrahman Mohammed A. ABDULAAL, Riyadh (SA)

(72) Inventor: Abdulrahman Mohammed A. ABDULAAL, Riyadh (SA)

(21) Appl. No.: 18/876,936

(22) PCT Filed: Jun. 22, 2023
(86) PCT No.: PCT/IB2023/056473

§ 371 Date: Dec. 19, 2024

Related U.S. Application Data
(60) Provisional application No. 63/354,503, filed on Jun. 22, 2022.

(51) Int. Cl. C25B 1/04; C02F 1/44; C02F 103/08; C07C 1/12; C25B 15/08

(52) U.S. Cl. CPC C25B 1/04; C02F 1/441; C07C 1/12; C25B 15/081; C25B 15/087; C02F 2103/08

(57) ABSTRACT

Systems and methods for producing hydrogen (H₂) from a desalination plant are described. The method can include desalinating saline water using energy produced by a gas turbine. Producing by splitting the desalinated water with an electrolyzer. The electrolyzer uses energy produced from the gas turbine to split the desalinated water. CO₂ can be captured from the gas turbine exhaust. Produced H₂ and captured CO₂ can be supplied to a reactor. In the reactor, a first product stream that includes H₂ and optionally methane (CH₄) can be obtained.

### CROSS REFERENCE TO RELATED APPLICATIONS

This application claims the benefit of priority of U.S. Provisional Patent Application No. 63/354,503, filed Jun. 22, 2022, which is hereby incorporated by reference in its entirety.

### BACKGROUND OF THE INVENTION

### A. Field of the Invention

The invention generally concerns production of hydrogen (H₂) from a desalination plant. The desalination plant can include a water desalination system, an electrolyzer, a gas turbine and a reactor for H₂ production. Desalinated water produced from the desalination system can be split into H₂ and O₂ with the electrolyzer. CO₂ from the gas turbine can be combined with the produced H₂ in a reactor to produce a first product stream that includes H₂ gas. Energy for the electrolyzer and the desalination plant can be produced from the gas turbine.

### B. Description of Related Art

Hydrogen (H₂) is an energy carrier. Its combustion can produce water as a byproduct. However, production of hydrogen can be energy inefficient and/or can produce undesirable by-products. Most of the hydrogen produced commercially is through steam methane reforming (SMR) of fossil fuels, which can release large amounts of CO₂ into the atmosphere. To decrease the amount of CO₂ emissions, natural gas is the primary source of the fossil fuel (“gray hydrogen”). However, this process also produces CO₂ albeit at a smaller rate than conventional SMR production. To decrease the CO₂ emissions, SMR can be coupled with carbon (e.g., CO₂) capture and/or carbon capture and storage (CSS). Hydrogen produced by this process can be referred to as “blue hydrogen”. Hydrogen produced by electrolysis from renewable energy-avoiding the release of climate-damaging CO₂ can be referred to as “green hydrogen”. However, the use of electrolyzer to produce hydrogen suffers from the need for pure water as a resource and high energy demands. To address the high energy demands, Boubenia et al., in “Carbone dioxide capture and utilization in gas turbine plants via integration of power to gas” (Petroleum, 2017, 3:127-137) describes energy sources for an electrolyzer and methanation reactor.

Overall, while the technologies of producing hydrogen exist, they can be energy inefficient and expensive.

### SUMMARY OF THE INVENTION

A discovery has been made that provides a solution to at least one of the problems associated with production of hydrogen. In one aspect, the discovery can include a system and method that produces a first product stream that includes hydrogen (H₂) with low to no CO₂ emissions by splitting desalinated water. The method can include using a gas turbine to provide energy to a desalinating system and an electrolyzer. The H₂ obtained from the electrolyzer can be combined with CO₂ from the gas turbine in a reactor to produce a first product stream that includes H₂. In some aspects, oxygen (O₂) can also be produced from the electrolyzer and can be stored, used as O₂, and/or used in other chemical reactions.

In one aspect, a method of producing H₂ gas from a water desalination plant is described. A method can include one or more steps. In step (a) desalinated water can be obtained by desalinating water that includes a solubilized salt and/or mineral with energy produced from a gas turbine. The desalinated water can be produced through reverse osmosis (RO), multistage flash desalination (MSF), multiple effect distillation (MED), or a combination thereof. The RO, MSF, or MED can include hollow fine fiber and spiral wound fiber. In some aspects, the solubilized salt and/or mineral can include solubilized sodium chloride. Non-limiting examples of water to be desalinated includes seawater, brackish water, or a combination thereof. In one aspect, water prior to desalination can include ions such as chloride ions (Cl⁻), sodium ions (Na+), sulfate ions (SO₄²⁻), magnesium ions (Mg²⁺), calcium ions (Ca²⁺), and/or potassium ions (K⁺), and the water after desalination includes less ions when compared with the water prior to desalination. Desalination can consume less than 3 kWh per cubic meter of water. In some aspects, desalination can produce at least 10,000, 50,000, 100,000, 150,000, 200,000, 250,000, 300,000, 350,000, 400,000, 500,000, 600,000, 700,000, 800,000, 900,000, 1,000,000, 2,000,000, 3,000,000, 4,000,000, 5,000,000, 6,000,000, 7,000,000, 8,000,000, 9,000,000, 10,000,000 cubic meters of desalinated water per day or any range or amount therein. In step (b), CO₂ gas can be obtained from exhaust produced from the gas turbine. In step (c) H₂ gas can be obtained by splitting the desalinated water of step (a) with an electrolyzer. Energy produced from the gas turbine used for desalination can be used by the electrolyzer. The electrolyzer can include a cathode, an electrode, and a membrane. In step (d) the H₂ gas obtained from water splitting can be combined with the CO₂ gas from the gas turbine in a reactor to produce a first product stream that includes H₂ gas. The reactor can be a methanation reactor. In some aspects, the reactor includes a methanation catalyst. The first product stream can be separated into at least a H₂ gas product stream and a CH₄ product stream. In some aspects, the first product stream can also include methane (CH₄), water, and/or CO₂. CO₂ from the first product stream can be recycled to the reactor. Water from the first product stream can be recycled to the electrolyzer.

In another aspect of the present invention water desalinization plants capable of producing hydrogen (H₂) gas and optionally oxygen (O₂) gas are described. A water desalinization plant can include (a) a water desalination system that includes a water desalinator, (b) a gas turbine for providing energy to the water desalination system (c) a carbon dioxide (CO₂) sequestering system for sequestering CO₂ gas from exhaust produced from the gas turbine, (d) an electrolyzer for splitting desalinated water and producing H₂ gas and optionally O₂ gas, where the electrolyzer can be operably connected to the water desalination system and the gas turbine, and (e) a reactor for reacting CO₂ gas from the exhaust and H₂ gas produced from the electrolyzer to produce a first product stream that includes H₂ gas. In one aspect, the water desalination system can include renewable power, H₂ storage systems, or a combination thereof. In another aspect, the electrolyzer can include one or more of a H₂ storage system, a heat recovering system, or a combination thereof. In some aspects, the reactor can include a methanation catalyst and a separation unit for separating the H₂ from the first product stream. The water desalinating plant can be capable of producing at least 400.000 cubic meters of desalinated water per day. In one aspect, the water desalination system can consumes less than 3 kWh per cubic meter of water. In some aspects, desalination plant can produce at least 10,000, 50,000, 100,000, 150,000, 200,000, 250,000, 300,000, 350,000, 400,000, 500,000, 600,000, 700,000, 800,000, 900,000, 1,000,000, 2,000,000, 3,000,000, 4,000,000, 5,000,000, 6,000,000, 7,000,000, 8,000,000, 9,000,000, 10,000,000 cubic meters of desalinated water per day or any range or amount therein. O₂ gas produced from the electrolyzer can also be produced. The produced O₂ gas from the electrolyzer can be stored, used as O₂, and/or used in other chemical reactions as a reactant.

Other embodiments of the invention are discussed throughout this application. Any embodiment discussed with respect to one aspect of the invention applies to other aspects of the invention as well and vice versa. Each embodiment described herein is understood to be embodiments of the invention that are applicable to other aspects of the invention. It is contemplated that any embodiment or aspect discussed herein can be combined with other embodiments or aspects discussed herein and/or implemented with respect to any method or composition of the invention, and vice versa. Furthermore, compositions of the invention can be used to achieve methods of the invention.

The following includes definitions of various terms and phrases used throughout this specification.

The terms “about” or “approximately” are defined as being close to as understood by one of ordinary skill in the art. In one non-limiting embodiment, the terms are defined to be within 10%, preferably within 5%, more preferably within 1%, and most preferably within 0.5%.

The terms “wt. %”, “vol. %”, or “mol. %” refers to a weight percentage of a component, a volume percentage of a component, or molar percentage of a component, respectively, based on the total weight, the total volume of material, or total moles, that includes the component. In a non-limiting example, 10 grams of component in 100 grams of the material is 10 wt. % of component.

The term “substantially” and its variations are defined to include ranges within 10%, within 5%, within 1%, or within 0.5%.

The terms “inhibiting” or “reducing” or “preventing” or “avoiding” or any variation of these terms, when used in the claims and/or the specification includes any measurable decrease or complete inhibition to achieve a desired result.

The term “effective,” as that term is used in the specification and/or claims, means adequate to accomplish a desired, expected, or intended result.

The use of the words “a” or “an” when used in conjunction with any of the terms “comprising,” “including,” “containing,” or “having” in the claims, or the specification, may mean “one,” but it is also consistent with the meaning of “one or more,” “at least one,” and “one or more than one.”

The words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

The processes and systems of the present invention can “comprise,” “consist essentially of,” or “consist of” particular ingredients, components, compositions, etc. disclosed throughout the specification. With respect to the transitional phrase “consisting essentially of,” in one non-limiting aspect, a basic and novel characteristic of the methods and/or systems of the present invention is their abilities to produce H₂ gas from desalinated water with an electrolyzer, where the electrolyzer uses energy produced from a gas turbine. The energy from the gas turbine can be used to run the desalination process and to run the electrolyzer.

Other objects, features and advantages of the present invention will become apparent from the following figures, detailed description, and examples. It should be understood, however, that the figures, detailed description, and examples, while indicating specific embodiments of the invention, are given by way of illustration only and are not meant to be limiting. Additionally, it is contemplated that changes and modifications within the spirit and scope of the invention will become apparent to those skilled in the art from this detailed description. In further embodiments, features from specific embodiments may be combined with features from other embodiments. For example, features from one embodiment may be combined with features from any of the other embodiments. In further embodiments, additional features may be added to the specific embodiments described herein.

### BRIEF DESCRIPTION OF THE DRAWINGS

Advantages of the present invention may become apparent to those skilled in the art with the benefit of the following detailed description and upon reference to the accompanying drawing.

FIG. 1 is an illustration of system and method to produce H₂ gas from a water desalination plant.

FIG. 2 is an illustration of a H₂ and other gases storage system.

While the invention is susceptible to various modifications and alternative forms, specific embodiments thereof are shown by way of example in the drawing. The drawing may not be to scale.

### DETAILED DESCRIPTION OF THE INVENTION

A discovery has been made that provides a solution to at least one of the problems associated with producing H₂ from a desalination plant. In one aspect, the discovery can include an energy efficient, environmentally sustainable system and method to produce H₂ by energy from a gas turbine to power a desalination system, an electrolyzer, and/or a CO₂ conversion reactor. In some aspects, the desalination system provides the advantage of being able to produce desalinated water at at least 400,000 cubic meters per day from seawater and/or brackish water while consuming less than 5, 4, 3, 2, or 1, or 0.5 kWh per cubic meter of water per day while having a low carbon footprint. In preferred aspects, the desalination system of the present invention can produce desalinated water at at least 400,000 cubic meters per day from seawater and/or brackish water while consuming 1 to 3 or 2 to 3 kWh per cubic meter of water per day.

These and other non-limiting aspects of the present invention are discussed in further detail with reference to the FIGURE.

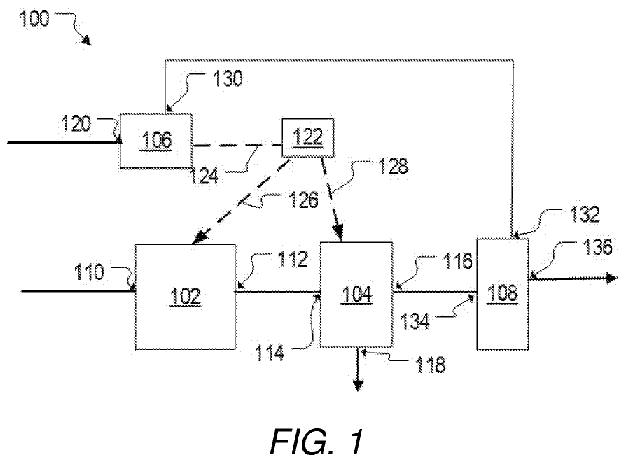

Referring to FIG. 1, a system and method for the production of H₂ from a water desalination plant 100 is described. Desalination plant 100 can include desalination system 102, electrolyzer unit 104, gas turbine 106, and reactor 108 (e.g., methanation reactor) in addition to other auxiliary units. Saline water (e.g., seawater or brackish water) can enter desalination system 102 via water inlet 110. Saline water can include solubilized ions and minerals (e.g., solubilized NaCl). Non-limiting examples of ions include chloride ions (Cl⁻), sodium ions (Na⁺), sulfate ions (SO₄²⁻), magnesium ions (Mg²⁺), calcium ions (Ca²⁺), and/or potassium ions (K⁺). In desalination system 102, the water can pass through one or more water purification units. Water purification units can be one or more reverse osmosis units, one or more multistage flash distillation units (MFD), one or more multiple effect distillation units (MED). In one aspect, a reverse osmosis unit having hollow fine fiber and/or spiral wound fiber can be used. As the saline water passes through the water purification unit, the solubilized salts and minerals in the saline water can be removed to produce desalinated water. The produced water exiting desalinating system 102 includes less ions when compared with the saline water entering the desalinating system. Desalination system 102 can produce at least 400,000 cubic meters of desalinated water per day (e.g., 400,000, 450,000, 500,000, or more) while consuming less than 3 kWh per cubic meter of water (e.g., 0.1, 0.5, 1, 1.5, 2, 2.5, 3 kWh per cubic meter of water per day). In other aspects, the desalination system 102 can produce at least 400,000 cubic meter of desalinated water per day (e.g., 400,000, 450,000, 500,000, or more) while consuming 3, 4, 5, 6, 7, 8, 9, 10, 11, 12, 13, 14, 15 kWh per cubic meter of water.

Produced desalinated water can exit desalination system 102 via desalination exit port 112, and enter electrolyzer unit 104 via electrolyzer inlet 114. Electrolyzer 104 can include a cathode (negative charge), an anode (positive charge), and a membrane. Electrolyzer 104 can also contain pumps, vents, storage tanks, a power supply, separator and other auxiliary components or equipment. In electrolyzer 104, electricity can be applied to the anode and/or cathode across the membrane (e.g., a proton exchange membrane (PEM)) and cause the desalinated water (H₂O) to split into its component molecules, hydrogen (H₂) and oxygen (O₂). A temperature of water splitting can be at least 10° C., 15° C., 20° C., 30° C., 40° C., 50° C. 60° C., 70° C., 80° C., 90° C., 100° C., or 110° C. or more (or any range or number therein). H₂ can exit electrolyzer via H₂ outlet 116 and be stored or provided to reactor 108. O₂ can exit electrolyzer 104 via O₂ outlet 116 and be stored or transported to other units for processing. In some embodiments a solid state electrolyzer can be used.

Desalination system 102 (e.g., water purification unit(s) and/or auxiliary equipment) and electrolyzer 104 can both powered by gas turbine 106. Electrolyzer 104 can be any commercially available electrolyzer to split water. A non-limiting example is the PEM Electrolyzer HyLYZER® Series 200-30 (Cummins, Inc., Columbus, Indiana U.S.A.). Other examples of electrolyzers include Electrolyzer HyLYZER® Series 250-30, 400-30, 500-30, 1000-30, and/or 4000-30. Gas turbine 106 can be any commercially available gas turbine sized to produce sufficient power for desalination system 102 and electrolyzer 104. Non-limiting examples of gas turbines that can be used include a GE Turbine (e.g., 50 Hz turbines (e.g., 9HA (448-571 MW), 9F (288 MW), GT13E2 (195-210 MW), 9E (132-147 MW), LMS100 (106.5-113 MW), 6F (57-88 MW), LM6000 (44.7-56 MW), 6B (45 MW), LM2500 (33-36.3 MW), and/or TM2500 (34.6 MW). Other examples of gas turbines can be 60 Hz GE Turbines (e.g., 7HA (290-430 MW), 7F (201-239 MW), LMS100 (107.5-115.8 MW), 7E (90 MW), 6F (57-88 MW), LM6000 (44.7-56 MW), 6B (45 MW), LM2500 (34.1-37.1 MW), and/or TM2500 (37 MW)). GE Turbines can be obtained from General Electric (GE) (Boston, Massachusetts, USA). Natural gas or methane can enter gas turbine 106 via gas inlet 120. In gas turbine 106, natural gas can be combusted to generate power and exhaust. Power can exit gas turbine 106 and enter power controller 122 via power line 124. Power controller 122 can store power generated by gas turbine 106 and distribute the generated power as needed to desalination system 102 and electrolyzer 104 via power lines 126 and 128, respectively. In another aspect, gas turbine 106 is directly coupled to desalination system 102 and electrolyzer 104 (not shown). In one aspect, reactor 108 is also powered by gas turbine 106. Exhaust from gas turbine 106, can include CO₂ and/or other gaseous combustion by-products. Gas turbine 106 can include a purification unit that removes combustion by-products from the CO₂. In some aspects, a purification unit is coupled to the gas turbine 106 (not shown). The purification unit can be any CO₂ purification unit. Non-limiting examples of purification units include a sorbent purification unit, a membrane separation unit, a cryogenic distillation unit, or a combination thereof. Non-limiting examples of sorbent purification can include solid adsorbents in pressure swing adsorption, temperature swing adsorption, or a sorbent/solvent unit. A gas separation unit can include gas separation membranes that allow CO₂ to pass through the membrane faster than the gaseous combustion by-products. Purified CO₂ can be stored or transported to other units for processing (e.g., reactor 108) via exhaust outlet 130.

Purified CO₂ can exit gas turbine 106 and enter reactor 108 via CO₂ inlet 132. H₂ generated from electrolyzer 104 can exit the electrolyzer via H₂ outlet 116 and enter reactor 108 via H₂ inlet 134. Reactor 108 can include any catalyst capable of catalyzing the reaction of CO₂ and H₂ to produce the first product stream that includes methane (CH₄), water (H₂O), and optionally unreacted starting materials H₂ and CO₂. The catalyst can include nickel ruthenium, rhodium, or an alloy or combination thereof. The catalyst can also include platinum, palladium, rhenium, iron, cobalt, vanadium, chromium, manganese, zirconium, an alloy thereof, or a combination thereof as additives. The catalyst can be supported (e.g., CeO₂, Al₂O₃, SiO₂, TiO₂ supports, or a combination or a mixture thereof) or unsupported. A non-limiting example of a reactor catalyst is a supported nickel catalyst. A temperature of reactor 108 can range from, 200° C. to 700° C. (e.g., 200° C., 300° C., 350° C., 400° C., 450° C., 500° C., 550° C., 600° C., 650° C., 700° C. or any value or range there between). In some aspects reactor 108 is a methanation reactor. Reactor 108 can include a separation unit and other auxiliary equipment. First product stream can exit reactor 108 via product outlet 136 and be stored, transported and/or further processed. For example, first product stream can be separated using known separation methods (e.g., distillation, membrane and the like) to produce a hydrogen stream, a methane stream, and a CO₂/H₂O stream. Methane stream can be stored, combined with gas turbine feed, or provided to gas turbine feed inlet 120. H₂ can be stored, combined with electrolyzer feed, or provided to electrolyzer feed inlet 114. CO₂/H₂O can be stored, separated or transported to another unit for processing. In some aspects, the CO₂ is separated from the H₂O using known methods (e.g., membrane separation) to produce a CO₂ stream that is provided to reactor 108.

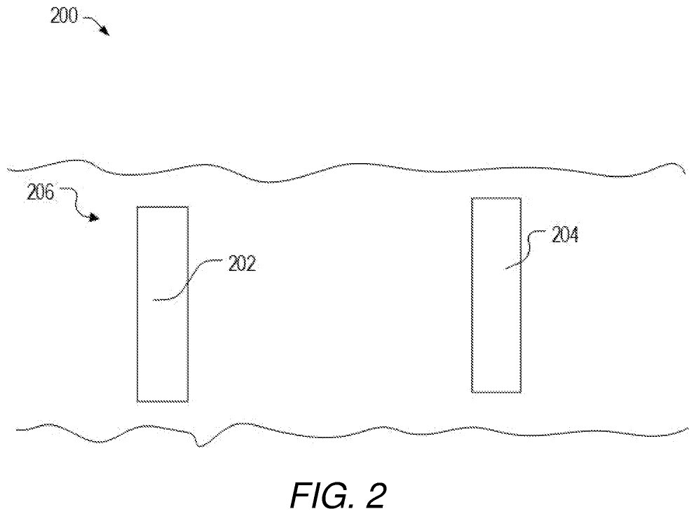

In one aspect, generated or recovered H₂ can be stored in a salt cavern. Referring to FIG. 2, salt cavern 200 can be constructed by dissolving underground salt deposits with water, and extracting the brine solution into a holding pond, thereby leaving one or more salt caverns in the formation (e.g., salt caverns 202 and 204 in formation 206). The salt cavern(s) can be lined to prevent leakage and potential contamination of aquifers. The H₂ generated from System 100 or other sources can be injected and stored into hydrogen salt cavern 202. The H₂ can stored indefinitely and then removed when needed. In other aspects, one or more salt caverns or veins can be prepared and other gases, for example, a methane stream generated in reactor 108 can be stored in salt cavern 204. The brine solution can be used as a feed source for the desalination plant.

In FIG. 1, desalination system 102, electrolyzer 104, gas turbine 106, and reactor 108 can include one or more heating and/or cooling devices, and control devices (e.g., valves, flow controllers and related instrumentation), inlets, outlets, etc. that can help control the temperatures and pressures of reactions or separations processes or movement of streams. It should be understood that one or multiple reactors can be housed in one unit. The temperature, pressure, and flowrate can be varied depending on the reaction to be performed and is within the skill of a person performing the reaction (e.g., an engineer or chemist).

### EXAMPLES

The present invention will be described in greater detail by way of specific examples. The following examples are offered for illustrative purposes only, and are not intended to limit the invention in any manner. Those of skill in the art will readily recognize a variety of noncritical parameters which can be changed or modified to yield essentially the same results.

### Example 1 (Prophetic Example)

A desalination system of the present invention can be implemented by using the scheme illustrated in FIG. 1 and optionally FIG. 2 and the information provided in the specification. An example of an electrolyzer that can be used in the context of the present invention to split water is the PEM Electrolyzer HyLYZER® Series 200-30 (Cummins, Inc., Columbus, Indiana U.S.A.). Other Electrolyzer HyLYZER® Series can also be used (e.g. Series 250-30, 400-30, 500-30, 1000-30, and/or 4000-30). The gas turbine that can be used can be a GE Turbine (e.g., 50 Hz turbines (e.g., 9HA (448-571 MW), 9F (288 MW), GT13E2 (195-210 MW), 9E (132-147 MW), LMS100 (106.5-113 MW), 6F (57-88 MW), LM6000 (44.7-56 MW), 6B (45 MW), LM2500 (33-36.3 MW), and/or TM2500 (34.6 MW) or 60 Hz turbines (e.g., 7HA (290-430 MW), 7F (201-239 MW), LMS100 (107.5-115.8 MW), 7E (90 MW), 6F (57-88 MW), LM6000 (44.7-56 MW), 6B (45 MW), LM2500 (34.1-37.1 MW), and/or TM2500 (37 MW)). The desalination system that can be used in the context of the present invention includes Saline Water Conversion Corporation (SWCC) (Riyadh, Saudi Arabia) systems. SWCC has a total of 32 desalination plants in 17 locations (e.g., Al-Khobar, Al-Jubail, Jeddah, Ras Al Khair Industrial City, etc.).

Although embodiments of the present application and their advantages have been described in detail, it should be understood that various changes, substitutions and alterations can be made herein without departing from the spirit and scope of the embodiments as defined by the appended claims. Moreover, the scope of the present application is not intended to be limited to the particular embodiments of the process, machine, manufacture, composition of matter, means, methods and steps described in the specification. As one of ordinary skill in the art will readily appreciate from the above disclosure, processes, machines, manufacture, compositions of matter, means, methods, or steps, presently existing or later to be developed that perform substantially the same function or achieve substantially the same result as the corresponding embodiments described herein can be utilized. Accordingly, the appended claims are intended to include within their scope such processes, machines, manufacture, compositions of matter, means, methods, or steps.

1. A method of producing hydrogen (H₂) gas from a water desalination plant, the method comprising:
(a) desalinating water comprising a solubilized salt and/or mineral with energy produced from a gas turbine to obtain desalinated water;
(b) obtaining carbon dioxide (CO₂) gas from exhaust produced from the gas turbine;
(c) obtaining H₂ gas by splitting the desalinated water with an electrolyzer, wherein the electrolyzer uses energy produced from the gas turbine to split the desalinated water; and
(d) contacting the H₂ gas with the CO₂ gas in a reactor to produce a first product stream comprising H₂ gas.

2. The method of claim 1, wherein the reactor is a methanation reactor, and wherein the first product stream further comprises methane (CH₄).

3. The method of claim 2, wherein the first product stream is separated into at least a H₂ gas product stream and a CH₄ product stream.

4. The method of claim 2, wherein the first product stream further comprises water and CO₂.

5. The method of claim 4, wherein CO₂ from the first product stream is recycled to the reactor.

6. The method of claim 4, wherein water from the first product stream is recycled to the electrolyzer.

7. The method of claim 1, wherein desalinated water is produced through a reverse osmosis unit, a multistage flash distillation (MSF) unit, a multiple effect distillation (MED) unit, or a combination thereof.

8. The method of claim 7, wherein step (a) consumes less than 3 kWh per cubic meter of water.

9. The method of claim 7, wherein step (a) produces at least 400,000 cubic meters of desalinated water per day.

10. The method of claim 7, wherein the reverse osmosis unit comprises hollow fine fiber and spiral wound fiber.

11. The method of claim 1, wherein the solubilized salt and/or mineral comprises solubilized sodium chloride.

12. The method of claim 1, wherein the water being desalinated comprises seawater and/or brackish water.

13. (canceled)

14. The method of claim 1, wherein the water prior to desalination comprises ions selected from chloride ions (Cl⁻), sodium ions (Na+), sulfate ions (SO₄²⁻), magnesium ions (Mg²⁺), calcium ions (Ca²⁺), and/or potassium ions (K⁺), and wherein the water after desalination comprises less ions when compared with the water prior to desalination.

15. The method of claim 1, wherein the reactor comprises a methanation catalyst.

16. The method of claim 1, wherein the electrolyzer comprises a cathode, an electrode and a membrane.

17. The method of claim 1, wherein oxygen (O₂) gas is obtained in step (c) by splitting the desalinated water with the electrolyzer.

18. A water desalinization plant capable of producing hydrogen (H₂) gas, the water desalinization plant comprising:
(a) a water desalination system comprising a water desalinator;
(b) a gas turbine for providing energy to the water desalination system;
(c) a carbon dioxide (CO₂) sequestering system for sequestering CO₂ gas from exhaust produced from the gas turbine;
(d) an electrolyzer for splitting desalinated water and producing H₂ gas, wherein the electrolyzer is operably connected to the water desalination system and the gas turbine; and
(e) a reactor for reacting CO₂ gas from the exhaust and H₂ gas produced from the electrolyzer to produce a first product stream comprising H₂ gas.

19. The water desalinization plant of claim 18, wherein the water desalination system comprises renewable power, H₂ storage systems or a combination thereof.

20. The water desalination plant of claim 18, wherein:
the electrolyzer comprises one or more of a H₂ storage system, a heat recovering system, or a combination thereof; and
the reactor comprises a methanation catalyst and a separation unit for separating the H₂ from the first product stream.

21. The water desalinization plant of claim 18, wherein the plant is capable of producing at least 400,000 cubic meters of desalinated water per day, and wherein the water desalination system consumes less than 3 kWh per cubic meter of water.
